(19) United States
(12) Plant Patent Application Publication
(10) Pub. No.: US 2004/0154062 P1
(43) Pub. Date: Aug. 5, 2004

(54) HYDRANGEA HYBRID PLANT NAMED 'SWEET CAROL'

(76) Inventor: Joseph Gray, Winters, CA (US)

Correspondence Address: CHRISTIE, PARKER & HALE, LLP, P.O. BOX 7068, PASADENA, CA, 91109-7068, US

(21) Appl. No.: 10/357,634

(22) Filed: Feb. 3, 2003

(51) Int. Cl. A01H 5/00

(52) U.S. Cl. PLT/250

(57) ABSTRACT

A new plant variety of Hydrangea hybrid characterized by its large lacecap inflorescence, which is reminiscent of its macrophylla lineage, while being substantially hardier than macrophylla cultivars; the white-eye zone on sterile inflorescences when young; deeply serrated leaves; and merlot colored fall foliage.

### BACKGROUND OF THE INVENTION

The present invention relates to a new and distinct variety of Hydrangea hybrid, which was developed in a controlled breeding program in Silver Spring, Md., United States, by Mr. Joseph Gray. The varietal denomination of the new variety is ‘Sweet Carol’.

The genus Hydrangea is included in the family Saxifragaceae which comprises about 80 genera and 1,200 species of herbs, shrubs, small trees and woody climbers mostly temperate in origin. Hydrangea comprises approximately 23 species of deciduous or evergreen shrubs and vines originating in North America, South America and East Asia, many of which possess desirable ornamental characteristics. Recent taxonomical treatments sometimes isolate Hydrangea in a new family, Hydrangeaaceae.

Hydrangea macrophylla is widespread and common throughout much of Japan. It generally has opposite, simple leaves, obovate to elliptic in shape, 10 to 20 cm long and 5 to 15 cm wide. Leaf color ranges from light to dark green, depending on light intensity and cultural conditions. Leaves are generally softly pubescent. Flower color is dependent upon the pH level of the soil and the amount of available aluminum in the soil media. Flowers of Hydrangea macrophylla are borne in mostly sterile florets, formed in what is often called a “hortensia” flower.

Hydrangea serrata is widespread and common throughout much of Japan, Korea and China. It generally has opposite, simple leaves, lanceolate in shape with acuminate apices, 5 to 15 cm long and 5 to 10 cm wide. Leaf color ranges from light to dark green, depending on light intensity and cultural conditions. Leaves are generally softly pubescent. The plant is deciduous. Flower color is dependent upon the pH level of the soil and the amount of available aluminum in the soil media. Flowers of Hydrangea serrata are borne in mostly flattened corymbs, 1 to 5 cm wide, with up to 12 sterile florets.

### SUMMARY OF THE INVENTION

The new variety was discovered in a controlled breeding program of Hydrangea serrata and H. serrata×macrophylla, and differs from its parents in its large lacecap inflorescence, which is reminiscent of its macrophylla lineage, while being substantially hardier than macrophylla cultivars; the white-eye zone on sterile inflorescences when young; deeply serrated leaves; and merlot colored fall foliage. Asexual reproduction of the new variety by stem cuttings, performed in Silver Springs, Md. and Vacaville, Calif., have confirmed that the distinctive characteristics of the new variety are stable and transmitted to succeeding generations, and the new variety reproduces true to type.

### COMPARISON WITH PARENTS

‘Sweet Carol’ is distinguished from its parents and all other varieties of Hydrangea of which I am aware in its large lacecap inflorescence, which is reminiscent of its macrophylla lineage, while being substantially hardier than macrophylla cultivars; the white-eye zone on sterile inflorescences when young; deeply serrated leaves; and merlot colored fall foliage.

### BRIEF DESCRIPTION OF ILLUSTRATIONS

The accompanying illustrations show a specimen of the new cultivar in a photo illustration as true to color as is reasonably possible to make in an illustration of this character.

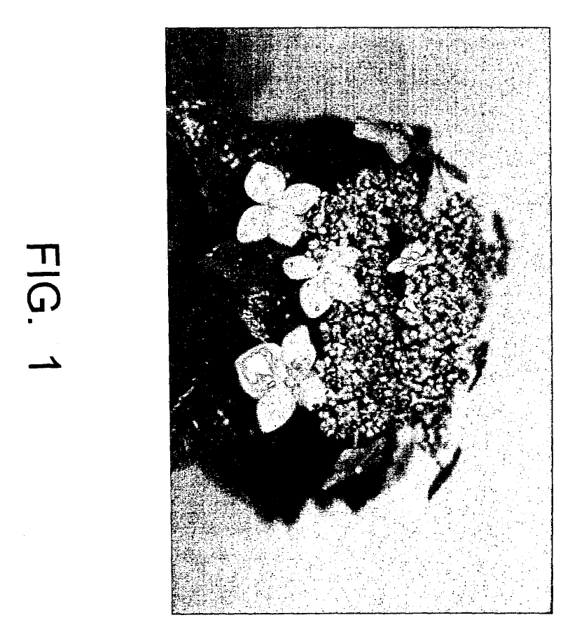

FIG. 1 illustrates the inflorescence and overall habit of ‘Sweet Carol’

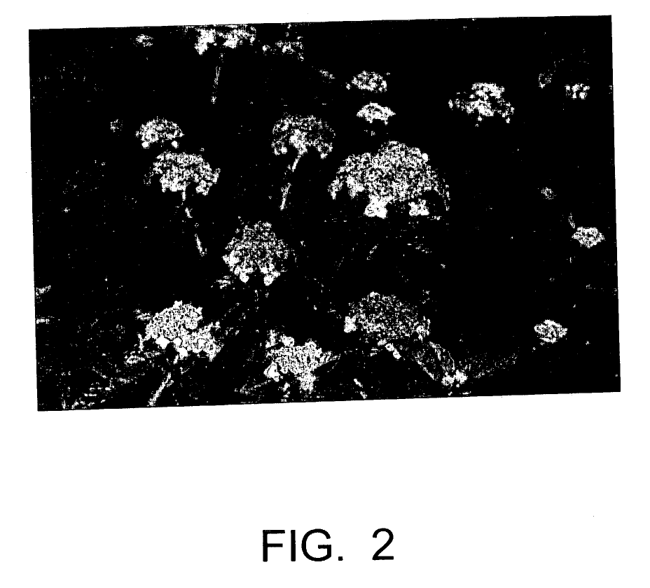

FIG. 2 illustrates the inflorescence of ‘Sweet Carol’

### DETAILED DESCRIPTION OF THE NEW VARIETY

‘Sweet Carol’ has not been observed under all possible environmental, cultural and light conditions. The following observations and descriptions are of plants grown in Vacaville, Callif. In this description, color references are to The Royal Horticultural Society Colour Chart (2001) and terminology used in the color descriptions herein refers to plate numbers in this color chart. Phenotypic expression may vary with light intensity, cultural and environmental conditions.

### CLASSIFICATION

Botanical: Hydrangea ‘Sweet Carol’.

Parentage:

Mother plant.—Hydrangea serrata×macrophylla ‘Lanarth White’.

Father plant.—Hydrangea serrata ‘Blue Billow’.

Propagation: By vegetative cuttings.

The following plant descriptors and color readings are of mature specimens grown in Vacaville, Calif. under 50% shade, in a 6.6 pH soiless nursery growing media.

### PLANT

Size: 1.5 to 2 meter tall.

Habit: Upright, compact shrub.

Branching: Approximately 30.

Lateral branches.—30.

Typical # branches.—20.

Observed # branches.—20.

Size.—2.5 meters.

Diameter.—7 mm.

Color of mature shoots.—Near Grey — Orange Group 177B.

Color of new shoots.—Near Green Group 143C.

### LEAF

Shape: Ovate to ovate lanceolate, acuneate to attenuate at the base and acuminate to long acuminate apices.

Leaf size: To 19 cm long and 8 cm wide.

Arrangement: Opposite.

Margin: Deeply serrated; serrations are up to 4 mm deep.

Substance: Thick, corriacious, to slightly succulent.

Texture:

Underside.—Glabrous, slightly glossy.

Upper side.—Glabrous, dull to slightly glossy.

Venation: Opposite or alternately pinnate.

Underside.—Raised.

Upper side.—Impressed.

Color.—New Foliage: Upper Surface: Near Yellow-Green Group 146B Under Surface: Near Yellow-Green Group 144B Mature Foliage: Upper Surface: Near Yellow-Green Group 146B Under Surface: Near Yellow-Green Group 144B.

Leaf color:

Young foliage.—Near Green Group 138B.

Mature foliage.—Upper side: Near Green Group 139 Underside: Near Green Group 138B-C.

Petioles:

Size.—2.5 to 3 cms long.

Coloration.—Near Green Group 138 B-C.

Texture.—Glabrous.

Internodes: 2 to 4 cm.

Hardiness: USDA Zone 6 (−10° F. to 0° F.).

Vigor: Vigorous growth.

Pests/Diseases: None noted.

### INFLORESENCE

Bloom period: May-August.

Inflorescence: Loose tiered corymb with both sterile (12), sepalous florets and fertile (650), non-sepalous florets borne on the same corymb.

Shape of inflorescence: Umbel of numerous small florets, sepalous florets are flat.

Inflorescence width: 14 to 20 cm across.

Flower arrangement: Loose tiered umbel 15 to 18 cm across (lace cap type).

Fragrance: None.

Lastingness of individual blooms: Flowers are very persistent, lasting up to 5 months. As the flowers mature, the pedicels bearing the outer florets bend to invert the florets toward the ground.

Infertile florets:

Sepals.—Usually 4, sometimes 5, sepals per floret, with one dominant sepal, 2 smaller equally sized sepals, and 1 yet smaller sepal.

Sepal size.—Dominant Sepal: To 30 mm wide and 22 mm long Mid-sized Sepal: Small Sepal: Number: Approximately 4 to 5 sepals per floret; usually 4. Deeply serrated with some entire. Usually one dominant sepal, 2 smaller but equally sized sepals, and one small sepal. Large sepals can measure as much as 30 mm wide and 22 mm long Shape: Ovate Size: Non-uniform; typical sepal measures 6 cms in diameter Margin: Entire except for 25% of margin at apex is dentately serrated Curvature: Abaxial Arrangement: Overlapping at base Color: Near Red-Purple Group 62B at soil pH of 6.6. However, sepals can be variable in color, depending on the soil pH.

Fertile florets.—Size of Floret: Approximately 1 mm in diameter Color of Floret: Near Red-Purple Group 63B.

### REPRODUCTIVE ORGANS

Androecium:

Stamens.—Approximately 7 to 10.

Stigma.—Usually 4, occasionally 3 or 5.

Stamen filament Color.—Near Red-Purple Group 57C.

Pollen amount.—Very little, not measurable.

Pollen color.—Near White Group 155D.

Gynecium:

Pistil.—Number: Usually 4; occasionally 3 or 5 Length: 2 mm.

Fertile inflorescence:

Pistil length.—2 mm.

Stigma color.—Near Red-Purple Group 57D.

Style color.—Near Red-Purple Group 57D.

Ovary color.—Near Red-Purple Group 57D.

Stamen filament.—Near Red-Purple Group 57C.

Fruit.—None noted.

I claim:

1. A new plant variety of Hydrangea hybrid of the variety substantially as shown and described.
